(19) United States
(12) Patent Application Publication
(10) Pub. No.: US 2015/0203803 A1
(43) Pub. Date: Jul. 23, 2015

(54) Device and Method for Bacterial Culture and Assay

(71) Applicant: National Tsing Hua University, Hsin Chu City (TW)

(72) Inventors: Jyun-Yu Lin, Kaohsiung City (TW); Hsi-Kai Wang, Taipei City (TW); Po-Hsien Chiu, New Taipei City (TW); Min-Yen Hsu, Taichung City (TW); Ying-Cheng Shen, Taichung City (TW); Chao-Min Cheng, New Taipei City (TW)

(21) Appl. No.: 14/339,287

(22) Filed: Jul. 23, 2014

(30) Foreign Application Priority Data
Jan. 20, 2014 (TW) 103101990

(51) Int. Cl. C12M 1/22; G01N 33/569; C12M 1/00

(52) U.S. Cl. CPC C12M 23/10; C12M 23/38; G01N 33/56911

(57) ABSTRACT

A device for bacterial culture and assay comprises a cover having a top surface and an upper wall; a plate having a bottom surface and a lower wall, wherein a closed space is formed between the cover and the plate by engaging the cover with and the plate; and a paper strip with a uniform material, and fixed to the cover, wherein the paper strip is used to absorb a desired liquid, and the closed space is used to contain a culture medium.

### FIELD OF THE INVENTION

The invention is relevant to a device and method for bacterial culture and assay, especially, for enzyme-linked immunosorbent assay (ELISA).

### DESCRIPTION OF THE PRIOR ART

An ELISA uses characteristics of specific binding between antigen and antibody to test the specimen. Because the ELISA can be performed to evaluate either the presence of antigen or the presence of antibody in a sample, it is a useful tool for determining serum antibody concentrations (such as with the HIV test or West Nile virus). The assay has also been applied in the food industry to detect potential food allergens such as milk, peanuts, walnuts, almonds, and eggs. In toxicology, ELISA can also be used as a rapid presumptive screen for certain classes of drugs. The three main ELISAs are sandwich ELISA, indirect ELISA, and competitive ELISA.

A Sandwich ELISA is used to detect a sample antigen. The first step is preparing a surface to which a known quantity of capture antibody is bound. Any nonspecific binding sites on the surface are blocked. The antigen-containing sample is applied to the plate. The plate is washed to remove unbound antigen. A specific antibody is added to bind to the antigen. Enzyme-linked secondary antibodies are applied as detection antibodies that also bind specifically to the antibody's Fc region (nonspecific). After the plate is washed to remove unbound antibody-enzyme conjugates, a chemical is added and converted by the enzyme into a color or into a fluorescent or electrochemical signal. The presence and quantity of an antigen are determined by measuring the absorbency, fluorescence, or electrochemical signal of the plate wells.

The steps of indirect ELISA are as follows. A buffered solution of the antigen to be tested is added to each well of a microtiter plate, where it is allowed to adhere to the plastic through charge interactions. A solution of nonreacting protein, such as bovine serum albumin or casein, is added to well any plastic surface in the well that remains uncoated by the antigen. The primary antibody is then added and binds specifically to the test antigen coating the well. This primary antibody could also be in the serum of a donor to be tested for reactivity towards the antigen. A secondary antibody is added, which binds to the primary antibody. Although an enzyme is often attached to the secondary antibody, the enzyme does not substantialy affect the binding properties of the antibody. The left side of the diagram shows a case in which the primary antibody itself is conjugated to the enzyme. A substrate for this enzyme is then added. Since the color of the substrate is often affected by the reaction with the enzyme, the color change indicates whether the secondary antibody has bound to primary antibody, which is a strong indication of an immune reaction of the donor to the test antigen. This reaction can be helpful in a clinical setting, and in research. The higher the concentration of the primary antibody present in the serum, the stronger the color change. Often, a spectrometer is used to give quantitative values for color strength.

A third use of ELISA is for competitive binding. After an unlabeled antibody is incubated in the presence of its antigen, the bound antibody/antigen complexes are added to an antigen-coated well. The plate is washed to remove unbound antibodies. The secondary antibody, which is specific to the primary antibody, is then added. The secondary antibody is coupled to the enzyme. After a substrate is added, the remaining enzymes elicit a chromogenic or fluorescent signal. The reaction is eventually stopped before signal saturation occurs.

However, culture and testing are performed separately in conventional ELISA, which increases the cost and the number of required devices. Therefore, conventional ELISA is not suitable for mobile and rapid detection.

In summary, a device for performing bacterial culture and assay suitable for mobile and rapid detection is needed.

### SUMMARY OF THE INVENTION

The invention solves the above problems by directly cultivating and detecting bacteria with a single device. Therefore, the invention reduces costs and enables mobile and rapid detection. Further, detection accuracy is increased by using paper strips instead of well plates as in conventional ELISA.

An embodiment of the invention provides a device for bacterial culture and assay comprising: a cover having a top surface and an upper wall; a plate having a bottom surface and a lower wall, wherein a closed space is formed between the cover and the plate by engaging the cover with the plate; and a paper strip with a uniform material, and fixed to the cover, wherein the paper strip is used to absorb a desired liquid and bacteria in the desired liquid, wherein the closed space is used to contain a culture medium to cultivate the bacteria.

Among them, the paper strip is adhered to the top surface of the cover, and the device is used to contain the contact lenses.

Further, the invention may comprise an inner cover fitting inside the cover for gripping edges of the paper strip and thus fixing the paper strip between the inner cover and the cover, wherein the inner cover has a hole to expose a center part of the paper strip.

Alternatively, an upper end of the paper strip is adhered to the top surface of the cover, and length of a lower end of the paper strip that is overhung exceeds the upper wall of the cover.

Further, the invention may further comprises: an inner cover fitting inside the cover for gripping an upper end of the paper strip and thus fixing the paper strip between the inner cover and the cover, wherein the inner cover has a hole to allow a lower end of the paper strip passing through the inner cover and length of the lower end of the paper strip that is overhung exceeds the upper wall of the cover.

Among them, the inner cover is a flat substrate; the upper wall of the cover includes an internal thread; and the lower wall of the plate includes an external thread, wherein the cover engages with the plate by the internal thread of the cover and the external thread of the plate, and the inner cover includes a wall with a internal thread; and the lower wall of the plate includes an external thread, wherein the inner cover engages with the plate by the internal thread of the inner cover and the external thread of the plate.

Another embodiment of the invention provides a method for bacterial culture and assay with the above-mentioned device comprising steps of: pouring culture broth into the plate; holding the cover and absorbing the desired liquid with the paper string of the cover; engaging the cover with the plate and immerse the paper strip into the culture broth; and disposing the device to make the paper strip horizontal to cultivate the bacteria.

The method of the invention further comprises steps of: after completion of culture, opening the cover, pouring the broth off, conducting enzyme-linked immunosorbent assay directly; adding an antibody reagent with specificity into the plate, and engaging the cover with the plate again; flipping the device; opening the cover and waiting until the desired liquid of the paper strip is half vaporized; adding a coloring agents; and determining presence of the bacteria and getting an analyzed value based on color change and color concentration on the paper strip.

### BRIEF DESCRIPTION OF THE DRAWINGS

The primitive objectives and advantages of the present invention will become apparent upon reading the following description and upon reference to the accompanying drawings in which:

FIG. 1A is a histogram illustrating the ratio of average brightness in the paper-based ELISA of the invention to that in a control group for each concentration of bacteria without cell lysis;

FIG. 1B is a histogram illustrating the ratio of light absorbance in the traditional ELISA of the prior art to that in a control group for each concentration of bacteria without cell lysis;

FIG. 1C is a histogram illustrating the ratio of average brightness in the paper-based ELISA of the invention to that in a control group for each concentration of bacteria with cell lysis;

FIG. 1D is a histogram illustrating the ratio of light absorbance in the traditional ELISA of the prior art to that in a control group for each concentration of bacteria with cell lysis;

FIG. 2A illustrates a device for bacterial culture and assay in ELISA based on an embodiment of the invention;

FIG. 2B illustrates a device for bacterial culture and assay in ELISA based on another embodiment of the invention;

FIG. 2C is an exploded view of the cover of the device shown in FIG. 2B;

FIG. 3A illustrates a device for bacterial culture and assay in ELISA based on a further embodiment of the invention;

FIG. 3B is an exploded view of the cover of the device shown in FIG. 3A;

FIG. 4 illustrates a device for bacterial culture and assay in ELISA based on a further embodiment of the invention;

FIG. 5 illustrates a device for bacterial culture and assay in ELISA based on a further embodiment of the invention; and

FIG. 6 illustrates a device for bacterial culture and assay in ELISA based on a further embodiment of the invention.

### DESCRIPTION OF THE PREFERRED EMBODIMENT

In order to fully understand the manner in which the above-recited details and other advantages and objects according to the invention are obtained, a more detailed description of the invention will be rendered by reference to the best-contemplated mode and specific embodiments thereof. The following description of the invention is made for the purpose of illustrating the general principles of the invention and should not be taken in a limiting sense; it is intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein. The terminology used in the description presented below is intended to be interpreted in its broadest reasonable manner, even though it is being used in conjunction with a detailed description of certain specific embodiments of the invention. Certain terms may even be emphasized below; however, any terminology intended to be interpreted in any restricted manner will be overtly and specifically defined as such in this detailed description section. Where the context permits, singular or plural terms may also include the plural or singular term, respectively. Moreover, unless the word “or” is expressly limited to mean only a single item exclusive from the other items in a list of two or more items, then the use of “or” in such a list is to be interpreted as including (a) any single item in the list, (b) all of the items in the list, or (c) any combination of items in the list.

Preferred embodiments and aspects of the invention will be described to explain the scope, structures and procedures of the invention. In addition to the preferred embodiments of the specification, the present invention is widely applicable in other embodiments.

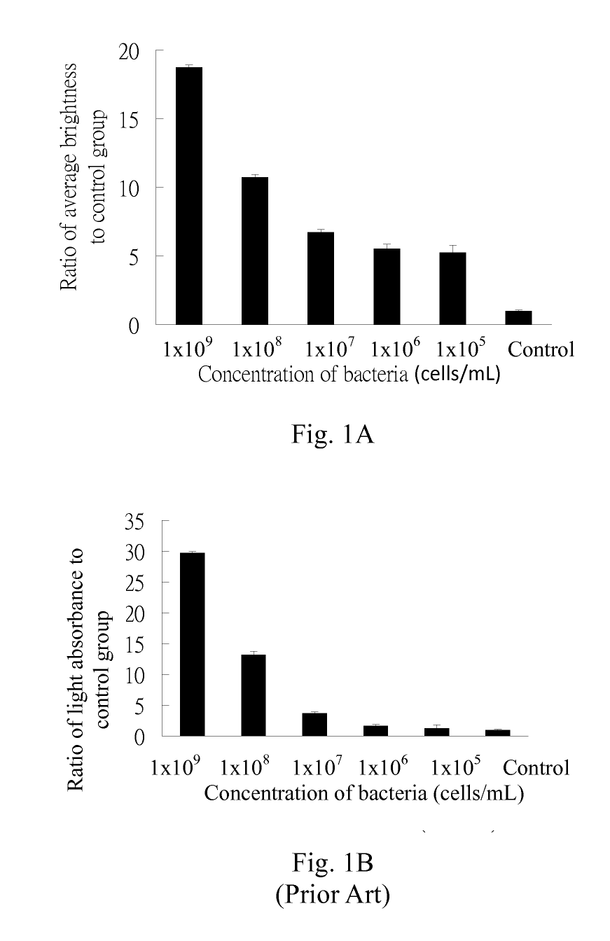

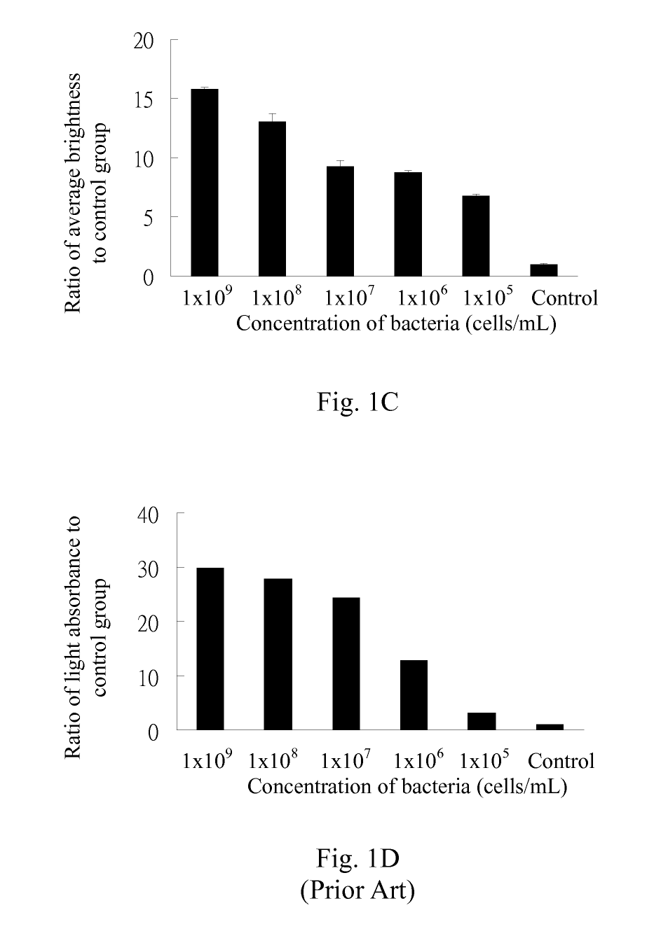

A sterile broth was used in the control groups (FIGS. 1A, 1B, 1C, and 1D). FIGS. 1A and 1C illustrate the results of the invention. The vertical axes indicates the ratio of average brightness in the paper-based ELISA of the invention to that in a control group. FIGS. 1B and 1D illustrate the results obtained by the prior art. The vertical axis indicates the ratio of average brightness in the conventional ELISA to that in a control group.

FIG. 1A is a histogram illustrating the ratio of average brightness (a.u.) in the paper-based ELISA of the invention to that in a control group for each concentration of bacteria without cell lysis.

FIG. 1B is a histogram illustrating the ratio of light absorbance in the conventional ELISA used in the prior art to that in a control group for each concentration of bacteria without cell lysis. FIG. 1B shows that the ratio of light absorbance in the experimental group to that in the control group decreases when the concentration of bacteria decreases.

Comparisons of the test results for the paper-based ELISA of the invention shown in FIG. 1A with those of the traditional ELISA shown in FIG. 1B prove that the paper-based ELISA of the invention is feasible. FIG. 1B shows that, when the concentration of cells is high (1×10⁹ cells/mL), the ratio of the experimental group to the control group is 30, which is better than the ratio of 18 obtained by of the paper-based ELISA of the invention. However, when the ratio is low (1×10⁵ cells/mL), the ratio of the paper-base ELISA of the invention increases to 5, which is better than ratio of 1.2 obtained by the traditional ELISA (FIG. 1B). Therefore, the paper-based ELISA in one embodiment of the invention has high sensitivity when the concentration is low, and the high sensitivity is an advantage of the present invention.

FIG. 1C is a histogram illustrating the ratio of average brightness (a.u.) in the paper-based ELISA of the invention to that in a control group for each concentration of bacteria with cell lysis. The ratio of light absorbance in the experimental group to that in the control group decreases when the concentration of bacteria decreases.

FIG. 1D is a histogram illustrating the ratio of light absorbance in the conventional ELISA of the prior art to that in a control group for each concentration of bacteria with cell lysis. FIG. 1D illustrates that the ratio of light absorbance in the experimental group to that in the control group decreases when the concentration of bacteria decreases.

Comparisons of the test results for the paper-based ELISA of the invention shown in FIG. 1C with those of the conventional ELISA shown in FIG. 1D confirm that the paper-based ELISA of the invention is feasible. FIG. 1D shows that, when the concentration of cells is high (1×10⁹ cells/mL), the ratio of the experimental group to the control group reaches 30, which is better than the ratio of 16 obtained by the paper-base ELISA of the invention. However, when the concentration is low (1×10⁵ cells/mL), the ratio obtained by the paper-based ELISA reaches 7, which is better than the ratio of 3 obtained by the conventional ELISA (FIG. 1D). The high sensitivity achieved by the paper-based ELISA of the invention when the concentration is low is an advantage of the present invention.

The conditions with and without cell lysis differ as follows: (1) when the concentration is high (1×10⁹ cells/mL), the ratios of the conventional ELISA reach 30, with or without cell lysis; (2) when the concentration is high (1×10⁹ cells/mL), the ratios of the paper-based ELISA of the invention are similar regardless of cell lysis; (3) when the concentration is low (1×10⁵ cells/mL), conventional ELISA with cell lysis obtains a slightly higher ratio compared to conventional ELISA without cell lysis; and (4) when the concentration is low (1×10⁵ cells/mL), the paper-based ELISA of the invention with cell lysis obtains a slightly higher ratio with cell lysis than it does without cell lysis. That is, the effectiveness of the paper-based ELISA platform of the present invention is unaffected by cell lysis.

Experimental culture and detection of Pseudomonas aeruginosa and Escherichia coli confirmed that the paper-based ELISA of the invention provide stable results with high sensitivity in small sample sizes and without the need for cell lysis. Further, the detection accuracy of the invention is increased by using well plates instead of the paper strips used in conventional ELISA.

In summary, the invention has the following advantages: (a) the inventions simplifies the required equipment, reduces costs, and improves ease of use because the paper-based bacterial culture and testing platform of the invention can be used not only to cultivate the bacteria, but also to test bacteria with the same device; (b) the paper-based bacterial culture and testing platform simplify the assay process and improve speed and efficiency because they can be used without blotting, withstand washing, and render the color of the detection result apparently even in the absence of or bacterial cell lysis or under low concentration; and (c) meeting the medical needs with market values. When treating corneal ulcers, samples must be taken for bacterial culture before using antibiotics. Among them, corneal ulcer caused by Pseudomonas aeruginosa have very poor treatment outcomes than other pathogens. Studies show that 6-39% of the United States population has Pseudomonas aeruginosa infection, which is highly toxic and has devastating effects on the eyes. Thus, rapid diagnosis and aggressive treatment are essential for preserving vision in these patients. Experimental culture and testing of Pseudomonas aeruginosa showed that the paper-based ELISA achieves faster screening compared to gel electrophoresis of the conventional ELISA. Based on the color reaction, Pseudomonas aeruginosa can be cultured in only 1 day, which increases the speed of diagnosis and enables early aggressive treatment.

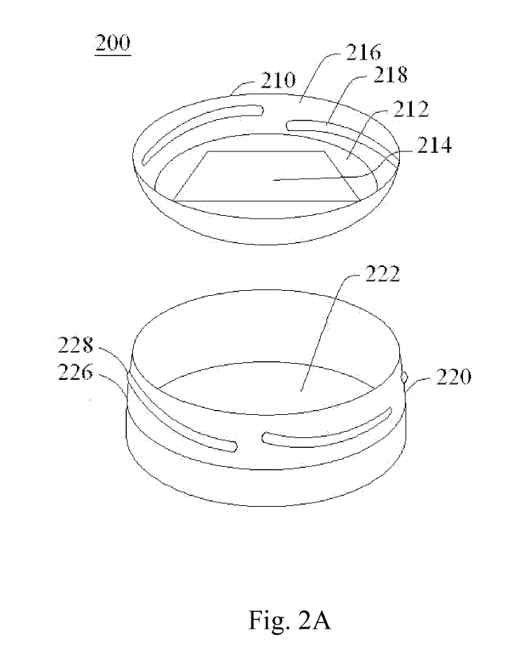

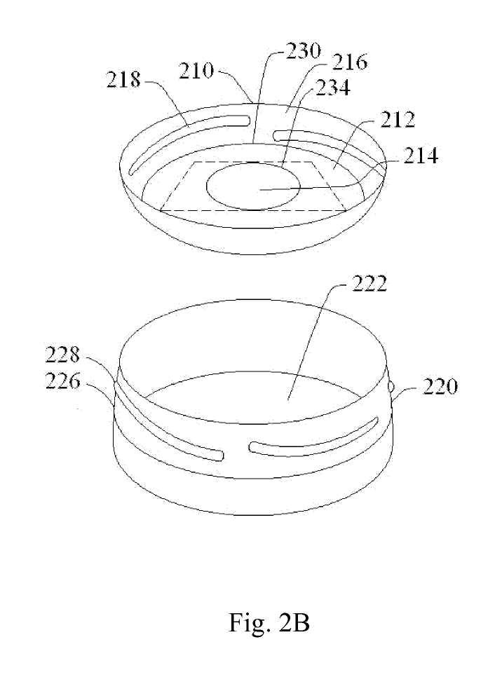

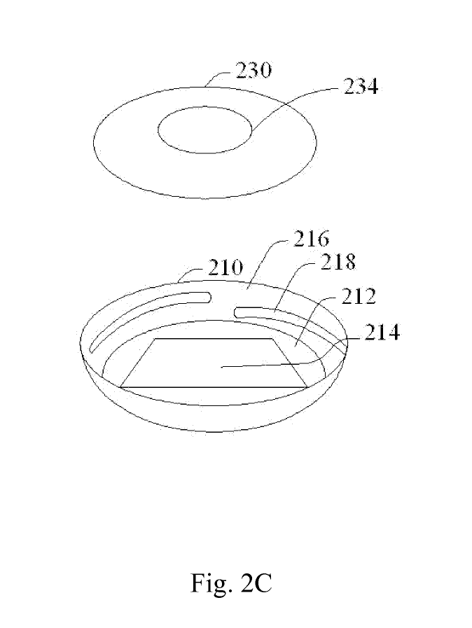

FIG. 2A is an ELISA device for bacterial culture and assay in one embodiment of the invention. Device 200 for ELISA bacterial culture and assay comprises a cover 210 with a top surface 212 and an upper wall 216; a plate 220 with a bottom surface 222 and a lower wall 226, wherein a closed space is formed between the cover 210 and the plate 220 by engaging the upper wall 216 of the cover 210 with the lower wall 226 of the plate 220; and a paper strip 214 composed of a uniform material and fixed to the cover 210, wherein the paper strip 214 is used to absorb a desired liquid and bacteria in the desired liquid, wherein the closed space is used to contain a culture medium to cultivate the bacteria. As shown in FIG. 2A, paper strip 214 is adhered to top surface 212 of cover 210.

FIG. 2B illustrates a device for bacterial culture and assay in ELISA based on another embodiment of the invention. FIG. 2C is an exploded view of the cover 210 and the inner cover 230 of the device shown in FIG. 2B. FIG. 2C shows an inner cover 230 fitted to the inside of the cover 210 for gripping the edges of the paper strip 214 to fix the paper strip 214 between the inner cover 230 and the cover 210. Further, the inner cover 230 has a hole 234 to expose a center part of the paper strip 214 from the inner cover 230.

FIG. 2C shows that the inner cover 230 is a flat substrate. The upper wall 216 of the cover 210 includes an internal thread 218; and the lower wall 226 of the plate 210 includes an external thread 228. Further, the cover 210 engages with the plate 220 by the internal thread 218 of the cover 210 and the external thread 228 of the plate 220.

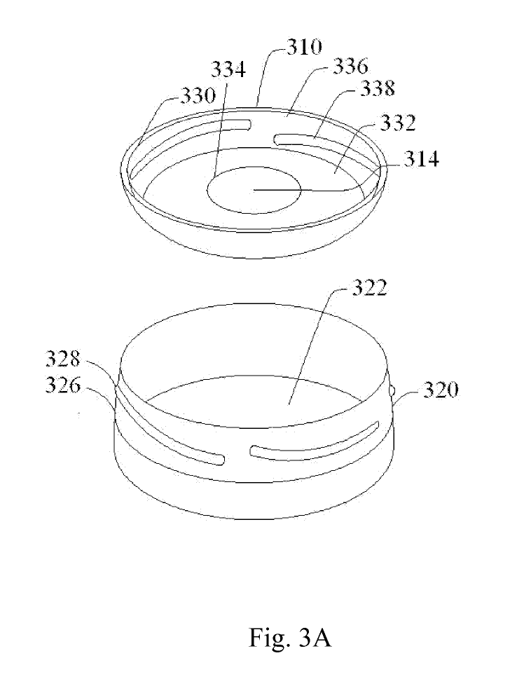

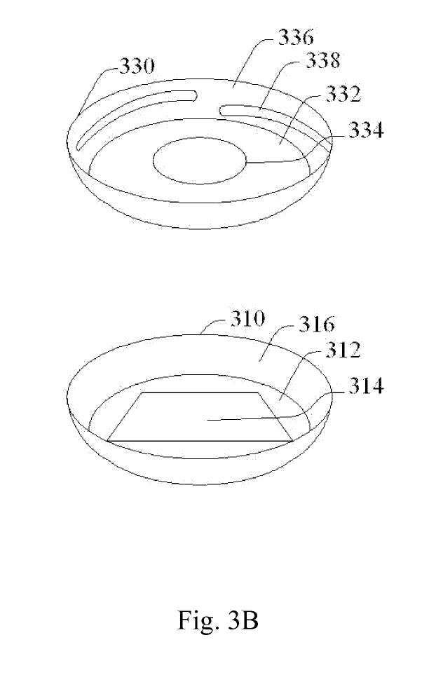

FIG. 3A is an ELISA device for bacterial culture and assay in another embodiment of the invention. FIG. 3B is an exploded view of the cover 310 and the inner cover 330 of the device shown in FIG. 3A. FIG. 3B shows that the inner cover 330 is fitted to the inside of the cover 310 to grip the edges of the paper strip 314 and to fix the paper strip 314 between the inner cover 330 and the cover 310. Further, the inner cover 330 has a hole 334 to expose a center part of the paper strip 314 from the inner cover 330.

FIG. 3B shows that the inner cover 330 comprises a wall 336. The wall 336 has an internal thread 338, and the lower wall 326 of the plate 310 includes an external thread 328. Further, the internal thread 338 of the inner cover 330 and the external thread 328 of the plate 320 are used to engage the cover 330 with the plate 338.

Alternatively, the bacterial cultivating and testing device can be a contact lens case. Before wearing contact lenses, the contact lens case can be filled with physiological saline, and the contact lenses can be soaked in the saline to determine whether the user has a bacterial infection in the eyes. This simplifies detection for contact lens wearers.

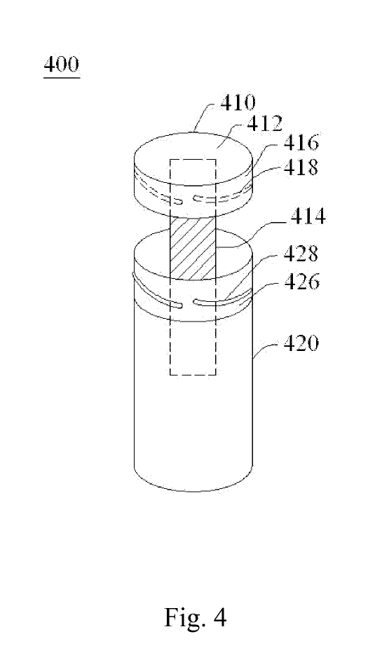

FIG. 4 is an ELISA device for bacterial culture and assay in another embodiment of the invention. The upper end of the paper strip 414 is adhered to the top surface 412 of the cover 410, and the length of the lower end of the paper strip 414 that is overhung exceeds the upper wall 416 of the cover 410. Therefore, a user can hold the cover 410 and use the lower end of the paper strip 414, which is overhung to absorb the desired liquid. Thus, the hands of the user are prevented from touching the paper strip 414 to contaminate the paper strip 414, which would affect the test results.

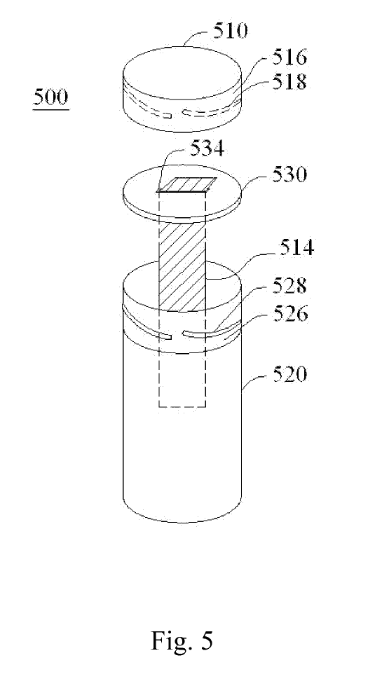

FIG. 5 is an ELISA device for bacterial culture and assay in another embodiment of the invention. The ELISA device 500 for bacterial culture and assay has an inner cover 530 fitted to the inner cover 510 for gripping edges of the paper strip 514 to fix the paper strip 514 between the inner cover 530 and the cover 510. The inner cover 530 has a hole 534, e.g., a slot, to allow the lower end of the paper strip 514 to pass through the inner cover 530, and the length of the lower end of the overhanging paper strip 514 exceeds the upper wall 516 of the cover 510.

FIG. 5 shows that the inner cover 530 is a flat substrate. The upper wall 516 of the cover 510 includes an internal thread 518; and the lower wall 526 of the plate 510 includes an external thread 528. Further, the cover 510 engages with the plate 520 by the internal thread 518 of the cover 510 and the external thread 528 of the plate 520.

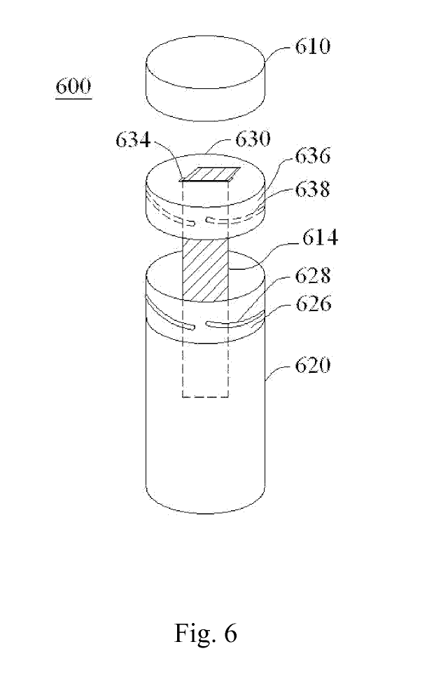

FIG. 6 illustrates an ELISA device for bacterial culture and assay based on another embodiment of the invention. An inner cover 630 is fitted to the inside of cover 610 to grip the edges of the paper strip 614 and to fix the paper strip 614 between the inner cover 630 and the cover 610. The inner cover 630 has a hole 634, e.g., a slot, so that the lower end of the paper strip 614 can pass through the inner cover 630, and length of the lower end of the paper strip 614 that is overhung exceeds the length of the upper wall 616 of the cover 610.

FIG. 6 shows that the inner cover 630 comprises a wall 636. The inner wall 636 has an inner thread 638, and the lower wall 626 of the plate 610 includes an external thread 628. Further, the internal thread 638 of the inner cover 630 and the external thread 628 of the plate 620 are used to engage the cover 610 with the plate 620.

The proposed method of bacterial culture and assay is performed in the following steps: (1) pour culture broth into the plate; (2) use the devices shown in FIGS. 4-6 to hold the cover and use the lower end of the overhanging paper strip to absorb the desired liquid. Thus, the user avoids handling the paper strip, which could cause contamination and affect the test results. Alternatively, the suction device can be used to draw the desired liquid if the devices shown in FIGS. 2A, 2B, and 3A are chosen; (3) engage the cover with the plate and immerse the paper strip in the culture broth; and (4) dispose the device so that the paper strip is horizontal to cultivate the bacteria. In other words, bacteria are cultivated by disposing the devices shown in FIGS. 4-6 horizontally and by disposing the devices shown in FIGS. 2A, 2B and 3A upside down.

When cultivation is completed, the following steps are performed to use the same device for testing: (1) open the cover, and pour off the broth; (2) add an antibody reagent with specificity to the bacteria into the plate, and re-engage the cover with the plate; (3) flip the device; and (4) open the cover and wait until the desired liquid of the paper strip is half vaporized; (5) add coloring agents; and (6) determine the presence of the bacteria and calculate an analysis value based on color change and color concentration on the paper strip.

In summary, the invention can be used to cultivate and test for bacteria with a single device. Therefore, the invention reduces costs and enables mobile and rapid detection.

The foregoing description was for purposes of explanation and was set forth in specific details of the preferred embodiments to provide a thorough understanding of the invention. However, it will be apparent to one skilled in the art that specific details are not required in order to practice the invention. Therefore, the foregoing descriptions of specific embodiments of the invention are presented for purposes of illustration and description only and should not be construed in any way to limit the scope of the invention. They are not intended to be exhaustive or to limit the invention to the precise forms disclosed; obviously, many modifications and variations are possible in view of the above teachings. The embodiments were chosen and described in order to best explain the principles of the invention and its practical applications, thereby enabling others skilled in the art to best utilize the invention and various embodiments with various modifications as are suited to the particular use contemplated. It is intended that the following Claims and their equivalents define the scope of the invention.

The above-described embodiments of the present invention are intended to be illustrative only. Numerous alternative embodiments may be devised by those skilled in the art without departing from the scope of the following claims.

1. A device for bacterial culture and assay comprising:
a cover having a top surface and an upper wall;
a plate having a bottom surface and a lower wall, wherein a closed space is formed between the cover and the plate by engaging the cover with the plate; and
a paper strip with a uniform material, and fixed to the cover, wherein the paper strip is used to absorb a desired liquid and bacteria in the desired liquid,
wherein the closed space is used to contain a culture medium to cultivate the bacteria.

2. The device of claim 1, wherein the paper strip is adhered to the top surface of the cover.

3. The device of claim 1, wherein the device is used to contain the contact lenses.

4. The device of claim 1, further comprising:
an inner cover fitting inside the cover for gripping edges of the paper strip and thus fixing the paper strip between the inner cover and the cover, wherein the inner cover has a hole to expose a center part of the paper strip.

5. The device of claim 1, wherein an upper end of the paper strip is adhered to the top surface of the cover, and length of a lower end of the paper strip that is overhung exceeds the upper wall of the cover.

6. The device of claim 1, further comprising:
an inner cover fitting inside the cover for gripping an upper end of the paper strip and thus fixing the paper strip between the inner cover and the cover, wherein the inner cover has a hole to allow a lower end of the paper strip passing through the inner cover and length of the lower end of the paper strip that is overhung exceeds the upper wall of the cover.

7. The device of claim 4, wherein
the inner cover is a flat substrate;
the upper wall of the cover includes an internal thread; and
the lower wall of the plate includes an external thread,
wherein the cover engages with the plate by the internal thread of the cover and the external thread of the plate.

8. The device of claim 4, wherein
the inner cover includes a wall with a internal thread; and
the lower wall of the plate includes an external thread,
wherein the inner cover engages with the plate by the internal thread of the inner cover and the external thread of the plate.

9. A method for bacterial culture and assay with the device of claim 5, comprising the steps of:
pouring culture broth into the plate;
holding the cover and absorbing the desired liquid with the paper string of the cover;
engaging the cover with the plate and immerse the paper strip into the culture broth; and
disposing the device to make the paper strip horizontal to cultivate the bacteria.

10. The method of claim 9, further comprising steps of:
after completion of culture, opening the cover, pouring the broth off, conducting enzyme-linked immunosorbent assay directly;
adding an antibody reagent with specificity into the plate, and engaging the cover with the plate again;
flipping the device;
opening the cover and waiting until the desired liquid of the paper strip is half vaporized;
adding a coloring agents; and
determining presence of the bacteria and getting an analyzed value based on color change and color concentration on the paper strip.

11. The device of claim 6, wherein
the inner cover is a flat substrate;
the upper wall of the cover includes an internal thread; and
the lower wall of the plate includes an external thread,
wherein the cover engages with the plate by the internal thread of the cover and the external thread of the plate.

12. The device of claim 6, wherein
the inner cover includes a wall with a internal thread; and
the lower wall of the plate includes an external thread,
wherein the inner cover engages with the plate by the internal thread of the inner cover and the external thread of the plate.

13. A method for bacterial culture and assay with the device of claim 6, comprising the steps of:
pouring culture broth into the plate;
holding the cover and absorbing the desired liquid with the paper string of the cover;
engaging the cover with the plate and immerse the paper strip into the culture broth; and
disposing the device to make the paper strip horizontal to cultivate the bacteria.

14. The method of claim 13, further comprising steps of:
after completion of culture, opening the cover, pouring the broth off, conducting enzyme-linked immunosorbent assay directly;
adding an antibody reagent with specificity into the plate, and engaging the cover with the plate again;
flipping the device;
opening the cover and waiting until the desired liquid of the paper strip is half vaporized;
adding a coloring agents; and
determining presence of the bacteria and getting an analyzed value based on color change and color concentration on the paper strip.
